(19) United States
(12) Patent Application Publication
(10) Pub. No.: US 2011/0060199 A1
(43) Pub. Date: Mar. 10, 2011

(54) DETERMINATION OF BLOOD PUMP SYSTEM PERFORMANCE AND SAMPLE DILUTION USING A PROPERTY OF FLUID BEING TRANSPORTED

(76) Inventors: Mark Ries Robinson, Albuquerque, NM (US); Mike Borrello, Carlsbad, CA (US); Richard P. Thompson, Dana Point, CA (US); Stephen Vanslyke, Carlsbad, CA (US); Shonn Hendee, Carlsbad, CA (US); Greg Knipstein, San Diego, CA (US); Dan Welsh, Encinitas, CA (US)

(21) Appl. No.: 12/884,175

(22) Filed: Sep. 16, 2010

Related U.S. Application Data
(63) Continuation-in-part of application No. PCT/US2009/037398, filed on Mar. 17, 2009.
Application No. PCT/US2009/037398 is a continuation-in-part of application No. PCT/US2009/037402, filed on Mar. 17, 2009.
Application No. PCT/US2009/037402 is a continuation-in-part of application No. 11/679,826, filed on Feb. 27, 2007.
Application No. 11/679,826 is a continuation-in-part of application No. PCT/US2006/060850, filed on Nov. 13, 2006.
(60) Provisional application No. 60/791,719, filed on Apr. 12, 2006.
(60) Provisional application No. 60/737,254, filed on Nov. 15, 2005.

(51) Int. Cl. A61B 5/157

(52) U.S. Cl. 600/316; 600/310; 600/309; 600/347; 600/365

(57) ABSTRACT

The use of an optical or other measurement in a blood access system enables the determination of a fluid sample appropriate for measurement on a real time basis. This information can be used to control the blood access system and related measurement processes. The determination can be based on, for example, at least one of: optical density, optical scatter, analyte level, temperature, the absolute level of any of the preceding, the stability of any of the preceding, the rate of change of any of the preceding, or the value of any of the preceding relative to another determination. The determination can be made using, for example, at least one of: electrochemical sensor, ion specific electrode, capacitance measurement, impedance measurement, inductance measurement, conductivity measurement, optical measurement, and ultrasound measurement. The present invention relates to determination of the quality of a biological sample in which determination of an analyte concentration is to be made, and various methods and apparatuses related thereto. An evaluation of sample quality can be made by monitoring the temporal changes in the sample properties or characteristics as the biological sample is procured or measured. The methods and apparatuses described herein can be used to evaluate the temporal characteristics of a sample during sample acquisition and/or during determination of the sample analyte or parameter of interest. The sample quality assurance methods and apparatuses described herein can thus be used to ensure that a valid sample has been procured by or presented to an instrument or measurement system for analyte determination, thereby preventing the measurement and reporting of analyte values for a sample that is unstable or otherwise non-representative of the biological system from which the sample was obtained.

### CROSSREFERENCE TO RELATED APPLICATIONS

This application claims priority as a continuation-in-part of PCT application PCT/US2009/037398, filed Mar. 17, 2009; and as a continuation-in-part of PCT application PCT/US2009/037402, filed Mar. 17, 2009; and as a continuation-in-part of U.S. application Ser. No. 11/679,826, filed Feb. 27, 2007, which application was a nonprovisional of U.S. provisional application 60/791,719, filed Apr. 12, 2006, and was a continuation-in-part of PCT application PCT/US2006/060850, filed Nov. 13, 2006, which PCT application claimed priority to U.S. provisional application 60/737,254, filed Nov. 15, 2005. Each of the preceding applications is incorporated herein by reference.

### TECHNICAL FIELD

The present invention relates to methods and apparatuses that withdraw blood or another fluid from a source such as a body, that can determine or compensate for mixing of the withdrawn fluid and the assisting fluid or can determination the quality of the sample.

### BACKGROUND ART

In creating a blood access system for measurement of blood analytes, the process generally involves removing the blood from the patient to a measurement site. The measurement is then made by a variety of methods and the blood is either discarded or re-infused into the patient. Access to the patient is typically through a catheter including, as examples, peripheral venous lines, PIC lines, arterial lines and central venous lines. In many cases, the access line between the patient and the pumping system is typically filled with a fluid, such as saline. It is common practice to infuse a small amount of saline between blood draws or measurements to help maintain the patency of the access site. This is referred to herein as a “keep vein open” or “KVO” rate. At the initiation of a draw the fluid-filled line reverses flow and blood is pulled toward the measurement site. The junction between the blood and the fluid is referred to as the “blood-fluid junction”; mixing of the fluid with the blood near the junction creates a “transition zone”. As the blood is drawn from the patient through the tubing, the blood/fluid interface exhibits a parabolic flow profile and is characterized by a broadened transition zone of blood mixed with fluid. Additional dilution can occur due to tubing discontinuities. The transition zone between undiluted blood and fluid increases in extent as the draw continues. Since analyte measurement systems are often sensitive to dilution effects, measurement accuracy can be enhanced by providing a sample for measurement that has a known or controlled dilution, for example a constantly diluted sample, a minimally diluted sample, or an undiluted sample, can facilitate accurate measurements. Hereafter the reference to an “undiluted” sample simply refers not only to a blood sample that has not been diluted but also to any sample that is suitable for accurate determination of blood analytes due to a known or controlled dilution characteristic. Accordingly, an “undiluted” sample can have dilution but of a quality that can be controlled, sensed, or managed. To obtain a blood sample representative of the blood in the patient, the blood access system can pull the diluted blood in the transition zone beyond the measurement site. Thus, the total amount of blood drawn is greater than the volume of the tubing between the measurement site and the patient. This dilution issue is known in the medical community and is generally addressed by drawing a discard sample or by filling an extra reservoir with diluted blood. As an example, the Edward's VAMP system includes such a reservoir.

In some systems, it can be desirable to also follow the sample with fluid so as to minimize the amount of blood that is removed from the patient. In this case, a second transition zone is created behind the undiluted sample.

In a system with defined and predictable operating characteristics, the withdrawal volume needed for procurement of an undiluted sample can be established and fixed. In most real-world blood access systems too many variables change over time and the system must have the capability of determining the presence of an undiluted sample. Some of the variables that change over time and between patients include:

Length and/or volume of the access catheter: central venous catheters generally have more volume and a longer length than peripheral catheters;

Extension tubing: the clinical staff might add extension tubing to the blood access system;

Blood viscosity changes due to differences in blood composition;

Blood hematocrit differences that influence the pressure needed to move the fluid and mixing characteristics at the blood-saline junction;

Pump tubing differences, including differences in internal volume and or pumping efficiency;

Pump efficiency changes over time.

Due to these and other variables that can change over time, the system must be able to determine the presence of an undiluted sample and then initiate the analyte measurement process.

Peristaltic pumps are commonly used in medical applications because they enable bidirectional pumping and can also prevent flow when the pump motor is not moving. However peristaltic pumps can be prone to pump volume differences between tubing sets and within a tubing set over time. In a peristaltic pump the volume accuracy is dependent on the volume captured between two or more occluding points, the pump rollers. The captured volume between the rollers is then propagated through the pump creating flow. For the pump to be accurate this captured volume must be constant. When a peristaltic pump withdraws fluid from a line there is a vacuum generated in the inlet of the pump. This vacuum can cause the tubing to collapse, and the captured volume between the occluding rollers will be less than in non-collapsed tubing. This can be compensated to an extent by monitoring the pressure at the inlet of the pump, and by adjusting the pump speed to withdraw the correct total volume. However, over time the tubing can fatigue so that it collapses more easily and the capture volume drifts down. As a result, the accuracy of the pump decreases over time. When withdrawing fluid from a line, the amount of fatigue varies from tubing set to tubing set and the change in fatigue varies increasingly over time (see, e.g., FIG. 1).

The determination of volume can be made with a flow meter. A number of ultrasonic flow meters are available commercially. By knowing the flow rate and the time period the amount of volume pumped can be determined. Volume determination helps to compensate for pump efficiency changes but does not completely compensate for blood changes. Additionally, such flow meters are expensive relative to overall system cost objectives.

For a blood access system designed to measure blood anatytes, the system should be able to determine when the fluid withdrawn is suitable for measurement. Due to the possibility of changing parameters associated with the blood being withdrawn, the physical volume of the blood access system and the efficiency of the pump system, the use of a fixed draw volume or draw time is inadequate. It can be desirable to minimize the total amount of blood withdrawn due to fluid infusion needs, the desire to remove from the patient as little blood as possible, and the desire to expose the tubing set to a minimum amount of blood over time.

Proper determination of an analyte for a biological system requires procurement or acquisition of a sample that is representative of the biological system prior to analyte determination. For example, measurement of blood analyte values and other blood parameters (such as blood counts, coagulation parameters, and oxygenation status) in patients usually requires that a blood sample be drawn from the patient for analysis. Caregivers frequently draw blood samples for analysis from arterial or venous access lines that are also used to infuse fluids to the patient. This generally requires that a volume of blood and fluid be pre-drawn from the access line to clear the line of the infusion fluid between the sample port and the tip of the catheter in the patient's vessel so that the desired measurement is performed on sample of blood and not on infusion fluid that may be still in the line. After the pre-draw is complete, the pure blood sample is drawn for analysis. When the pre-draw is not performed or is of insufficient volume to completely clear the line of the non-blood fluid, the blood sample that is procured for analysis can contain an unknown amount of the infusion fluid. The result is a sample that provides an erroneous result, either due to simple dilution (in the case where the infusion fluid is simple saline) or due to a false change in the analyte or parameter of interest due to the contamination of the sample by the constituents of the infusion fluid. Errors of this type that are associated with sample procurement prior to analyte or parameter determination are known in the clinical community as pre-analytical errors, and are among the most common errors encountered in measurements of blood chemistry and other biological fluid samples. Such errors can result in the need to repeat tests, causing delays in making medical decisions or administering treatment. In some cases, such errors can lead to erroneous medical decisions, leading to serious and sometimes even fatal medical consequences for the patient.

In addition to dilution or contamination of a blood sample by infusion fluid due to insufficient volume of pre-sample, there are several other situations that can compromise the quality of the biological sample. Examples include:

Acquisition of a blood sample simultaneously with administration through an adjacent vascular access line of a therapeutic agent or fluid. This can cause acquisition of a non-representative sample if the blood sample were drawn before the fluid were evenly distributed and equilibrated throughout the systemic blood volume. Acquisition of a sample during administration of a fluid or agent can be contaminated with the co-infused substance.

Administration of large volume physiological therapy, such as blood transfusion or blood volume expanders. As before, a blood sample drawn during such therapy can be an unstable or nonrepresentative sample.

It can be desirable to determine the quality of the sample prior to making the determination of the analyte or parameter of interest of the biological sample, thereby preventing the reporting of analytical values that have pre-analytical error due to improper or inadequate sample procurement or acquisition.

### DISCLOSURE OF INVENTION

The following description describes illustrative embodiments and is not intended to limit the scope of the invention. As used in this specification and the appended claims, the singular forms “a”, “an”, and “the” include plural referents unless the context clearly dictates otherwise. Thus, for example, reference to a method of classifying “a biological sample” includes a method of classifying more than one biological sample regardless of source. As used in this specification and the appended claims, the term “or” is generally employed in its sense including “and/or” unless the context clearly dictates otherwise.

The use of an optical measurement in the blood access system enables the determination of a fluid sample appropriate for measurement on a real time basis. This information can be used to control the blood access system and related measurement processes. The optical measurement system can take a variety of forms, including light emitting diodes and detectors, spectrometers, and interferometers. Wavelength regions of relevance can span from the ultraviolet to the far infrared. The visible, near infrared and mid infrared spectral regions can be of particular interest.

An optical measurement system can be used to determine when a sample appropriate for measurement has been procured. A suitable optical measurement system can determine the presence of fluid and blood and the relative ratio of each. Therefore, the system can determine when an appropriately undiluted sample is present at the measurement site. A suitable optical measurement system can make such an assessment in a number of ways.

Several are listed below:

Optical density: since blood is typically more optically dense than the diluting fluid, when a undiluted sample has arrived the optical density will approach a maximum or steady state level.

Optical scatter assessment: blood scatters light whereas fluids such as saline do not. When an undiluted sample is present, a scattering measure will approach a maximum or steady state level.

Detection of blood arrival: since fluid looks optically and spectroscopically different from blood, the system can sense the arrival of an initial amount of blood (in a diluted sample) and then estimate the time for an undiluted sample.

Analyte stability: the optical system can measure an analyte that is present in the blood and determine when stability of this measurement has occurred. The analyte can be present in both the IV fluid and blood but in different concentration. Analytes available for measure include but are not limited to glucose, protein, hemoglobin, cholesterol, albumin, urea and cholesterol.

Temperature differences: the blood will be at body temperature while the fluid may be at room temperature. This temperature difference can be used for determination of a suitable sample.

Optical information: any optical information that enables the detection of fluid versus blood can be used to determine the relative concentration of fluid versus blood, and the relative concentration to correct a measurement, or used to indicate when a suitable relative concentration is present at the measurement site.

The optical measurement method can be based upon a variety of measurement methodologies, as examples including absorption spectroscopy, Raman spectroscopy, fluorescence spectroscopy, atomic absorption spectroscopy, attenuated total reflectance spectroscopy, electron paramagnetic spectroscopy, electron spectroscopy, gamma-ray spectroscopy, infrared spectroscopy, laser spectroscopy, mass spectrometry, and x-ray spectroscopy.

In addition to optical measurements, other types of measurements can be used to determine when an undiluted sample is available for measurement. As examples, such measurements can be made by electrochemical sensors, ion specific electrodes, capacitance measures, impedance measures, inductance measures and conductivity measures. Additionally, a system that can sense particle matter can also be used. Ultrasound is an example of such a sensing system. Potassium concentration can be used as an illustrative example. Most intravenous fluids do not contain potassium. A real time measurement of potassium can be used to determine the presence of a suitable sample. The potassium measurement will be effectively zero at the start of the draw and then increase to a maximum as an undiluted sample arrives at the sensing location.

As blood is highly variable based upon the patient's condition, the above metrics can be used in an absolute sense, a relative sense, or rate of change sense. For example, optical density is influenced by sample hematocrit and can be difficult to implement in an absolute sense. Hematocrit can vary from 20% to 55%. Thus, an optical density measure of a hematocrit measurement can be more effectively implemented via a rate of change assessment. When the rate of change becomes small, the blood sample is no longer diluted and an undiluted sample is present. At this point in time a measurement can be made. Effective processing of the response signal will improve the robustness of the measurement. Optimal filtering of the signal for the estimation of the derivative can result in a more robust output. An appropriate filtering method can include one or more of the following characteristics: (1) provide good derivative estimation at low frequencies, (2) reject high frequency noise, and (3) minimize distortions at the key transition frequencies. Examples of filters with these types of characteristics are those known in the art as roof-top filters.

The invention disclosed is not dependent upon the measurement method used and is applicable to indwelling electrochemical sensors, enzymatic sensors, sensors that work when in contact with blood, such as those made by Dexcom and Abbott, standard sensors that work on a sample of blood and other optical sensing methods that use serum, plasma, or ultra filtrate. Additionally, the method can work on any fluid-sample junction. Examples of such possible junctions include saline-serum, saline-plasma, and saline-ultra filtrate, and saline-supernatant from a centrifuged sample.

An evaluation of sample quality can be made by monitoring the temporal changes in the sample properties or characteristics as the biological sample is procured or measured. The methods and apparatuses described herein can be used to evaluate the temporal characteristics of a sample during sample acquisition and/or during determination of the sample analyte or parameter of interest. The sample quality assurance methods and apparatuses described herein can thus be used to ensure that a valid sample has been procured by or presented to an instrument or measurement system for analyte determination, thereby preventing the measurement and reporting of analyte values for a sample that is unstable or otherwise non-representative of the biological system from which the sample was obtained.

As an example, FIG. 13 comprises plots of measurements of a property of biological samples. In the “Typical sample” plot, the sample parameter exhibits a variability over time expressed by one measure as 7.3. In the “High variance sample”, the sample parameter exhibits a variability over time expressed by one measure as 24. As can be seen from the plot, the high variability is caused by noise in the property measurement; there is no apparent dependence of the sample parameter on time. The high variability can indicate that the sample is inconsistent with the expected measurement environment, e.g., if a contaminant or an external noise source or sensor malfunction is causing the measured sample property to oscillate over time. The high variability can indicate a sensor system that is not operating as expected. In any case, a conventional point measurement would not detect the likelihood that the measurement was in error, while analysis of the variability of the property over time in accord with the present invention can reveal that the accuracy of a measurement of the high variance sample is likely to be less than the accuracy of a measurement of the typical sample.

As another example, FIG. 14 comprises pots of measurements of a property of biological samples. In the “Typical sample” plot, the sample parameter exhibits a variability over time expressed by one measure as 7.3. In the “Trending sample”, the sample parameter exhibits a variability over time expressed by on measure as 41.1. As can be seen from the plot, the high variability is caused at least in part by a decrease in the sample parameter over time. The high variability can indicate that the sample is inconsistent with the expected measurement environment, e.g., if a contaminant is causing the sample property to change over time, or the sample is becoming more or less diluted over time, or the sensor performance is degrading. In any case, a conventional point measurement would not detect the likelihood that the measurement was in error, while analysis of the variability of the property over time in accord with the present invention can reveal that the accuracy of a measurement of the trending sample is likely to be less than the accuracy of a measurement of the typical sample.

### BRIEF DESCRIPTION OF DRAWINGS

The figures filed herewith are intended to form part of the description, and help explain the present invention and example embodiments thereof.

FIG. 1 is a plot of peristaltic pump withdrawal volume under various operating conditions.

FIG. 2 is a schematic illustration of an example blood access system.

FIG. 3 is an illustration of blood flow into a saline-filled flowcell.

FIG. 4 is a flow diagram of an example optical termination operation.

FIG. 5 is a plot of a linear predictor (Bhat) for blood concentration in a blood saline mixture (0 to 100% blood).

FIG. 6 comprises plots illustrating glucose accuracy comparison between YSI and measurement using an optical termination method.

FIG. 7 is a schematic illustration of an example system that incorporates a parameter sensor to evaluate sample quality during acquisition or measurement of a biological sample.

FIG. 8 is a plot of an example of a parameter monitored continuously during sample acquisition with normal parameter variance.

FIG. 9 is a plot of an example of a parameter with a time trend during sample acquisition with normal parameter variance.

FIG. 10 is a schematic illustration of an example measurement system suitable for use with the present invention.

FIG. 11 is a flow diagram of a measurement cycle according to an example embodiment of the present invention.

FIG. 12 is a schematic illustration of measurement cycle metrics according to an example embodiment of the present invention.

FIG. 13 comprises plots of a sample parameter in a typical sample and in a sample with high variance.

FIG. 14 comprises plots of a sample parameter in a typical sample and in a sample with a trending in the value.

FIG. 15 is a plot of a parameter response exhibiting excessive noise without trending.

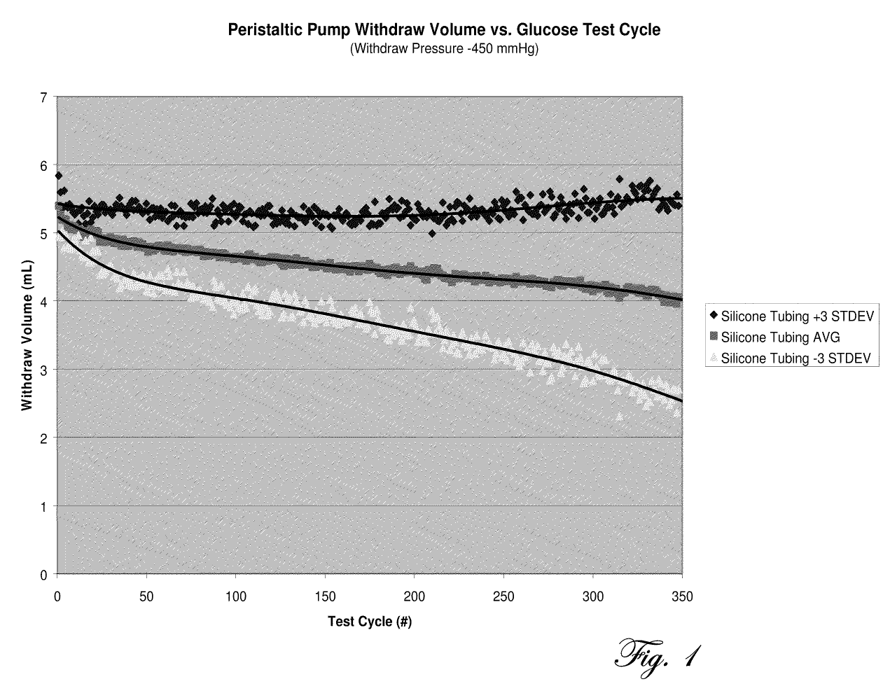

### DESCRIPTION OF THE INVENTION

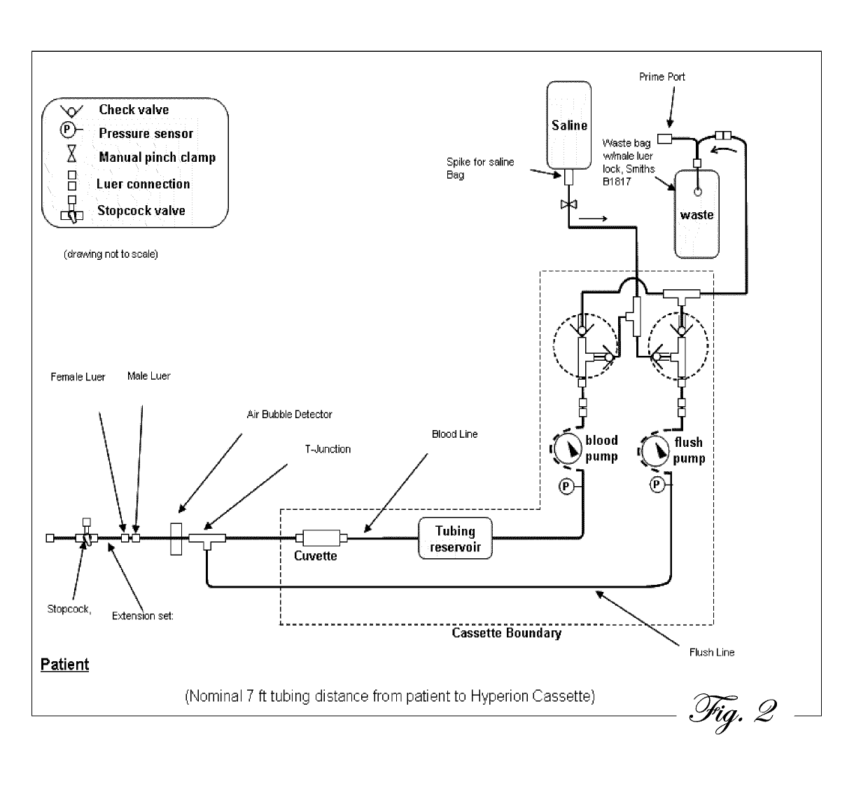

The ability to determine the presence of a suitable sample has been demonstrated and implemented on an example blood access system. The example blood access system was used to embodiments of the present invention; the present invention can be used with various other blood access systems and measurement techniques. The example system is used for convenience to explain operation of embodiments of the present invention in connection with a particular example system; the present invention is applicable to many other sample access and sample measurements systems, including as further examples those described in U.S. provisional applications 60/791,719, 60/913,582, 60/991,373, 61/044,004, 61/104,252, 60/991,447, and 60/992,037, 61/037,177, 61/037,315; PCT application PCT/US06/60850; U.S. application Ser. Nos. 11/679,826, 11/679,837, 11/679,839, 11/679,835, 10/850,646, 11/842,624, 12/188,205, 12/108,250, 12/325,243; each of which is incorporated herein by reference. The example blood access system is shown in FIG. 2, and can be described by considering three main component groups: 1) pump and measurement console, 2) a disposable sensor set, and 3) fluid bags that attach to the circuit.

The console can be attached to the patient through a sterile disposable sensor set designed for use with the console. As an example, the sensor set can be intended for use on a single patient for up to 72 hours. The sensor set, which can be attached to the patient using a dedicated peripheral venous catheter or other access location, provides convenient vascular access that enables automated withdrawal of a whole blood sample into an in-line optical cuvette for glucose measurement by means of optical transmission spectroscopy. When a glucose measurement is made, the system withdraws blood into the sensor set under controlled flow and pressure conditions. The system maintains flow of the blood during the glucose measurement, and reinfuses at least a portion of the blood to the patient once the glucose measurement is complete. The sensor set is connected to a saline bag which provides a flushing solution that keeps the lines and catheter free of thrombus formation and blood accumulation. In addition, the sensor set has a second path that connects to a waste bag through a T-junction near the patient connection. This path to waste enables thorough flushing and cleaning of the system between measurement cycles without infusing excess fluid to the patient.

The Console comprises:

Pumps—Pumps provide the ability to move blood and saline between the patient and the optical cuvette. There are two peristaltic pumps, the blood pump and the flush pump, that execute a programmed flow control sequence for the procurement of a blood sample for measurement, reinfusion of the blood following measurement and thorough cleaning of the sensor set after reinfusion. The sampling sequence is initiated by a manual request or pre-programmed, for example at a frequency or interval specified by the user.

Control System—Electronic controls and software manage pump speeds and directions and monitor the sensor set pressures during the blood measurement cycle. The blood measurement cycle will 1) maintain patency between blood samples, 2) withdraw a blood sample, 3) return the blood sample, and 4) clean the sensor set. If the Control System detects fault events in the blood access cycle, the control system will either execute automated procedures to clear the faults, or it will alert the user when faults cannot be automatically cleared. The Console also contains the optical measurement system, consisting of a light source and spectrometer for making the NIR glucose measurement. Glucose measurement algorithms can be resident in system nonvolatile memory.

Touch Screen—The Console can incorporate a touch screen computer for entering patient information and setting device operation parameters. The Console also provides visual display of measured glucose values as well as information associated with system operation including visual and audible alerts and alarms.

The Sensor Set includes:

Circuit Tubing—There are two tubes extending to the patient from the Cassette. One tube is used to convey blood and saline between the patient and optical cuvette. A second tube to the patient aids catheter flushing and returns saline used to clean the optical cuvette to the sensor set waste bag. Within the Cassette are a number of one-way valves used to isolate returned waste fluid from the patient.

Extension Set—The extension set connects the patient catheter to the disposable sensor set. The extension set includes a stopcock for lab blood draws and catheter maintenance, provides strain relief for ease of use and patient safety, and facilitates the attachment of the automated glucose measurement system to the patient.

Pump Cassette—The cassette attaches directly to the console and includes all electrical connections, peristaltic pump loops and one-way valves needed for operation. The cassette components comprise:

Pressure Sensors—Measure pressures inside the sensor set in the proximity of the pump tubing. There are two pressure sensors: the blood line pressure sensor and the flush line pressure sensor. Each sensor measures pressures on the patient side of the pump.

Tubing Reservoir—As a blood sample is withdrawn from the patient to the cuvette the first portion of the sample is diluted with saline. The diluted blood is pumped past the cuvette into the Tubing Reservoir. This overdraw enables measurement of an undiluted blood sample in the optical cuvette. The Tubing Reservoir is comprised of a vertical coil of tubing.

Bubble detector—The Blood Access System has a bubble detector that detects the presence of bubbles in the sensor set near the Extension Set. The bubble detector is used to ensure patient safety and to improve overall system functionality.

Cuvette—a glass tube with rectangular cross-section and fixed path length in which the blood measurement is made. The cuvette provides the interface between the sensor set and the spectrometer in the Optical Measurement System.

Two Fluid Bags can be useful for system operation:

Saline bag (user-supplied)—The Blood Access System pumps are able to move blood by pumping a column of sterile saline in advance of the blood sample. The sensor set accordingly requires a connection to a sterile saline bag. The sensor set is designed so that either the blood or flush pumps can pump fluid from the saline bag. One way valves ensure fluids cannot be pumped into the saline bag.

Waste bag—The Blood Access System requires a waste bag for collection and disposal of waste fluid generated during the flush and cleaning cycles. The sensor set is designed so that either the blood pump or flush pump can pump fluid into the waste bag. One way valves ensure fluids cannot be pumped out of the waste bag.

The Glucose Measurement System determines the blood glucose concentration by using an optical measurement based on near-infrared (NIR) spectrum of the blood sample. The measurement occurs by transmitting NIR light through a optical cuvette containing blood to produce a transmission spectrum. A Fourier transform infrared (FTIR) spectrophotometer can be used to collect NIR spectra. Specially designed software and hardware convert the spectra to glucose information which is reported on the console display.

### Operation of an Example Embodiment

From an operational standpoint, the instrument can be separated into two primary functional subsystems that work in tandem to achieve the automated glucose measurement: 1) Blood Access System and 2) Optical Measurement System. The role of the Blood Access System is to safely and reliably draw a homogeneous blood sample from the patient into the optical cuvette, maintain the sample in a stable condition during the course of the optical measurement, return the blood to the patient and then flush and prepare the system for the next measurement cycle. The role of the optical measurement system is to collect NIR transmission spectra from the blood contained within the sensor set cuvette and to apply the appropriate signal conditioning and spectral data processing to confirm that an undiluted sample is present in the cuvette and to make a glucose determination from that sample.

The Blood Access System (see FIG. 2) can deliver an undiluted blood sample from the patient to the optical measurement system at a distance of approximately 7 feet from the patient. The system initiates a blood draw, pulls the blood from the patient and through the optical cuvette for glucose measurement, then reinfuses the blood to the patient following the measurement cycle. The system addresses the following issues:

Procurement of an undiluted blood sample for optical measurement;

Minimization of blood loss and fluids infusion;

Continued patency of the catheter, tubing and optical cuvette.

Procurement of an undiluted blood sample for optical measurement. The automated blood access system can use a sensor set that is primed with saline for safe and effective blood flow control. As the blood is drawn from the patient through the tubing, the blood/saline interface exhibits a parabolic flow profile and is characterized by a broadened transition zone of blood mixed with saline. The transition zone between undiluted blood and saline increases as the draw continues. Since the glucose measurement system can be sensitive to dilution effects, diluted blood is drawn past the glucose sensor and collected in the tubing reservoir until an undiluted sample is present in the cuvette. The present invention can be used to determine when an appropriate sample is present in the optical cuvette. Upon arrival of an appropriate sample, the system can initiate the measurement process. In the example embodiment, the measurement system is an optical measurement system but other measurement methods can be used. Other suitable methods can include indwelling electrochemical sensors, enzymatic sensors, sensors that work when in contact with blood such as those made by Dexcom and Abbott, standard sensors that work on a sample of blood and other optical sensing methods that use serum, plasma, supernatants or ultrafiltrates.

Minimization of blood loss and fluids infusion. Because of the blood-saline mixing at the interface between the two fluids, reinfusion of blood can involve some saline infusion to the patient. Similarly, when the system diverts fluid to waste during the cleaning process there can be an amount of residual blood in the tubing that goes to waste with the flush solution. There is a tradeoff between the amount of saline infused to the patient with each cycle versus the amount of blood diverted to waste. The automated Blood Access System can provide an optimized balance to minimize blood loss while simultaneously minimizing the saline infused to the patient with each sample. Typical standard maintenance intravenous fluid infusion rates are 125 mL/hr (3.0 liters per day) for a typical sized person. The procurement of automated measurements every 30 minutes would result in 48 paired measurements over the 24 hour period. If each measurement cycle infuses 9 ml of saline to the patient this will represent approximately 15% of a typical fluid maintenance rate. To minimize saline infusion during the measurement cycle and subsequent cleaning requires careful monitoring of infused volume to compensate for blood-saline mixing, and the use of specific fluid flow rates and patterns that optimize cleaning of the tubing during the blood infusion and cleaning. In regular operation, the only blood that is lost is that which is cleared from the walls of the tubing into the waste bag during the flush cycle. The amount of blood lost is less than 1004 per sample or approximately 5 mL/day at a 30 minute sample interval.

Patency of the catheter, tubing and cuvette. Stationary extracorporeal blood, unless treated with anticoagulants, tends to adhere to foreign surfaces and coagulates within a few minutes. To avoid these issues the process of blood withdrawal, measurement, reinfusion and cleaning can be completed effectively within a time frame that prevents blood coagulation and achieves effective cleaning of the circuit so aggregation of blood components within the walls of the tubing, cuvette and catheter of the sensor set does not occur.

The plumbing network (see FIG. 2) contains check valves configured to allow saline to be drawn from the saline bag into either the blood or flush line, and waste fluids to be pumped through either of these lines into the waste bag. The valves prevent the system from drawing fluid from the waste bag or from pumping fluid into the saline bag. Both the blood pump and the flush pump can provide flow in either direction. For example, during infusion saline is pulled from the saline bag and flows toward the patient. During withdrawal, fluid from the blood line is pumped towards the waste bag. The pumps can be operated independently or together at matched, opposite or different flow rates. Independent clockwise rotation from either pump causes blood to be drawn from the patient towards that pump and counterclockwise rotation causes fluid to be infused into the patient from either pump. Since the blood line and flush line are connected to each other and to the patient through a “T” near the patient, if the blood pump is operated in a counterclockwise direction and the flush pump is operated in a clockwise direction at a matched rate, then fluid will flow from the blood line into the flush line, pulling saline from the saline bag and pumping it into the waste bag.

Exception Detection and Management. Exceptions to the normal operation of the automated glucose measurement system occur when occlusions and air bubbles appear during the operation of the Blood Access System. The Blood Access System detects and manages occlusions, restrictions and air bubbles that can occur during any phase of the operational cycle. The system utilizes different recovery methods depending upon the stage of operation. Using measurements from the two pressure transducers near the pumps, the system can identify the location of a problem and will automatically clear the problem or alert the user so that it can be cleared manually. If the exception requires the user to take an action this is called an intervention.

In the operation of a Blood Access System, interventions that can occur include:

Occlusions due to positional occlusions of the catheter;

Air bubbles (typically from saline out gassing) when the system cannot automatically flush them to waste.

The automated glucose measurement system can use the following information for occlusion detection and management:

Pressure thresholds based upon the stage of operation;

Relationship of pressure between the two pressure sensors;

The time history of the pressure relationships between the pressure sensors;

The time history of pressure measurements (trend changes);

Dissipation of pressure within the circuit (the pressure change between the withdrawal and sample stages);

Time to complete a stage or time to complete stages;

Pressure trends between subsequent withdrawals;

Estimated flow rates based on pump rotational speeds and differential pressure readings.

This information can be incorporated into a decision flow chart that determines if an occlusion has occurred and initiates an appropriate recovery process. Generally, the system determines the stage of operation, the presence of blood in the circuit, the location of the occlusion and implements a recovery process to the extent possible. Depending upon the recovery results, an operator such as a nurse can be alerted. For example if an occlusion occurs in withdrawal, the system automatically re-infuses any blood withdrawn and a small amount of additional saline. The system will re-attempt a second blood draw. If occlusion is detected a second time the system again re-infuses any blood removed and automatically returns to a safe condition and alerts the care provider to address the problem.

The example system can also detect air bubbles in the line and prevent them from being infused to the patient. Common causes of air bubbles include out-gassing of the saline as it is subjected to negative pressure, and an increase in ambient temperature compared to the storage temperature of the saline. The automated glucose measurement system detects air bubbles below the T-junction near the Extension Set and stops flow upon detection. The system then determines the stage of operation, and the presence of blood in the circuit. Based upon this information a bubble management protocol is initiated. In most cases the bubble is pulled from the air bubble detector and past the T-junction into the flush line. Once isolated in the flush line, the system can flush the bubble to the waste bag for disposal. The system then resumes normal operation and provides an alert to an operator such as a nurse.

### Detailed Description of Blood Access Operation in an Example Embodiment

The Blood Access System operation can be described as 6 primary stages:

Draw initialization and clearing the catheter access;

Blood withdrawal;

Optical measurement;

Infusion;

Cleaning (incorporating Scrub, Recirculation, and Catheter Flush sub-stages);

KVO (“keep vein open”).

Draw Initialization Stage; Clearing Catheter Access. Before the blood draw is started, both the blood and flush pumps are controlled to issue a pulse of saline to clean away any residual blood in the catheter tip. This prepares the catheter for the subsequent withdrawal of blood.

Blood Withdrawal Stage. The blood pump is used to withdraw the blood sample and position non-diluted blood in the cuvette. To minimize the total draw time, about 80% of the total required blood volume is first drawn at a rapid flow rate. A constant-pressure-based draw method is used to compensate for the varying mix of saline and blood, and to achieve maximum flow rate constrained by the constant upstream negative pressure that keeps fluid degassing minimized. As blood replaces saline in the blood line, viscosity and resistance to flow increase so that for a constant upstream pressure, flow rate decreases over time. The termination of this stage of the draw is determined by what is referred to as optical termination. Optical termination is the optical detection of when a sample appropriate for measurement has filled the cuvette. After the optical termination of the withdrawal stage, the measurement of the sample can be initiated. An example of a specific optical termination method will be disclosed in detail below. Non-optical methods of detecting the arrival of an undiluted blood sample, such as those described elsewhere herein, can also be used.

Optical Measurement Stage. Following the rapid draw, the pump flow rate is slowed to a constant flow rate of 0.5 mL/min to maintain suspension of the red blood cells in plasma during optical measurement. During the 60 second measurement period an additional 500 μl of blood is withdrawn.

Infusion Stage. After the measurement is completed, reinfusion immediately begins as a progression of stages that are designed to return the blood quickly to the patient and clean the tubing and optical cuvette. The initial stage of infusion uses a constant pressure-based control which results in a variable flow rate that minimizes the time to reinfuse the blood to the patient. This stage reinfuses nearly all of the blood that was withdrawn, leaving a remaining saline-blood mixture at the end of the blood line. The first stage of the reinfusion can be completed within three minutes of the initiation of blood withdrawal.

The 2nd stage of infusion involves a repetitive back and forth motion of the blood pump such that during half of one cycle the pump pushes blood forward at a constant flow rate, and during the second half of the cycle blood is pulled back at about half the rate. The asymmetric cycle helps wash away any cells or other blood products that could potentially adhere to the tubing walls. During this stage of infusion, flow is controlled to limit the pressure.

The 3rd stage of infusion begins with the blood pump executing a repetitive alternating forward-pause motion that provides pulsatile acceleration and washing of blood products from the tubing walls. The flow in this stage is also pressure controlled.

It is possible to use another optical termination type measurement to determine when the majority of blood has been re-infused back into the patient and exited the optical cell. The basic principles are the same but in this application the termination measurement is looking for stability in the saline sample instead of stability in the blood sample. The method can be used to make sure there is no residual blood in the cell.

Cleaning Stages. At this point in the cycle more than 97% of the blood has been returned to the patient; the next stages focus on a more thorough cleaning of the cuvette, tubing and catheter.

Scrub Stage. The first stage of cleaning is known as ‘scrub’. The scrub stage involves rapid, reverse-synchronized back and forth motion of the blood and flush pumps so that fluid movement occurs only within the blood and flush lines with minimum net fluid flow to or from the patient. The flow is not turbulent, but the rapid oscillations create accelerations that help to wash any small amount of residual blood that can collect on the walls of the tubing and cuvette.

Recirculation Stage. Once the remaining blood products have been lifted off the tubing and cuvette walls into the mainstream by the scrub stage, the blood and flush pumps are operated at a constant, nearly synchronized rate, flushing the lines into the waste bag while flushing a small amount of saline to the patient to keep blood from migrating back into the catheter.

It is also possible to use an optical termination type measurement to access when the cell has been adequately cleaned. Even small amounts of protein can be assessed optically. Thus, the optical measurement method can be used to determine when adequate cleaning of the cell has occurred. In use the method can compare the spectral response from a prior measurement to the current measurement. If there is optical evidence of additional protein in the cell then additional cleaning might be indicated.

Catheter Flush Stage. In the final cleaning stage high flow rate controlled volume pulses completely clear the catheter extension line, tubing connectors and the catheter itself.

KVO Stage. The period between measurement cycles is KVO (Keep Vein Open). KVO provides a low, constant flow rate into the patient to prevent blood from migrating into the catheter thus maintaining an open blood access connection between draws.

### Optical Termination Example

Optical Termination allows the use of an optical measurement through a flowcell or cuvette to determine the portion of blood in a blood-saline mixture; alternatively this method can be used to determine the portion of saline in a flowcell or cuvette (also referred to as an inverse termination) in near real-time. The blood access system operates by the use of a wash solution of saline, which during the draw cycle will produce a liquid phase wavefront that mixes with blood as the two materials come in contact during a standard blood draw and measurement sequence.

A requirement of knowing the precise concentration of blood within the flowcell at all times during a blood draw sequence can be complex due to high pump rates (required for short draw times) and where the actual flow rate is a function of pump tubing wear, roller position at the start of the draw, and blood viscosity or blood access limitations. The minimum volume required for a precise glucose recovery can change with catheter volume (central venous versus peripheral), and unintended variation in tubing lengths or diameters added to the sensor set.

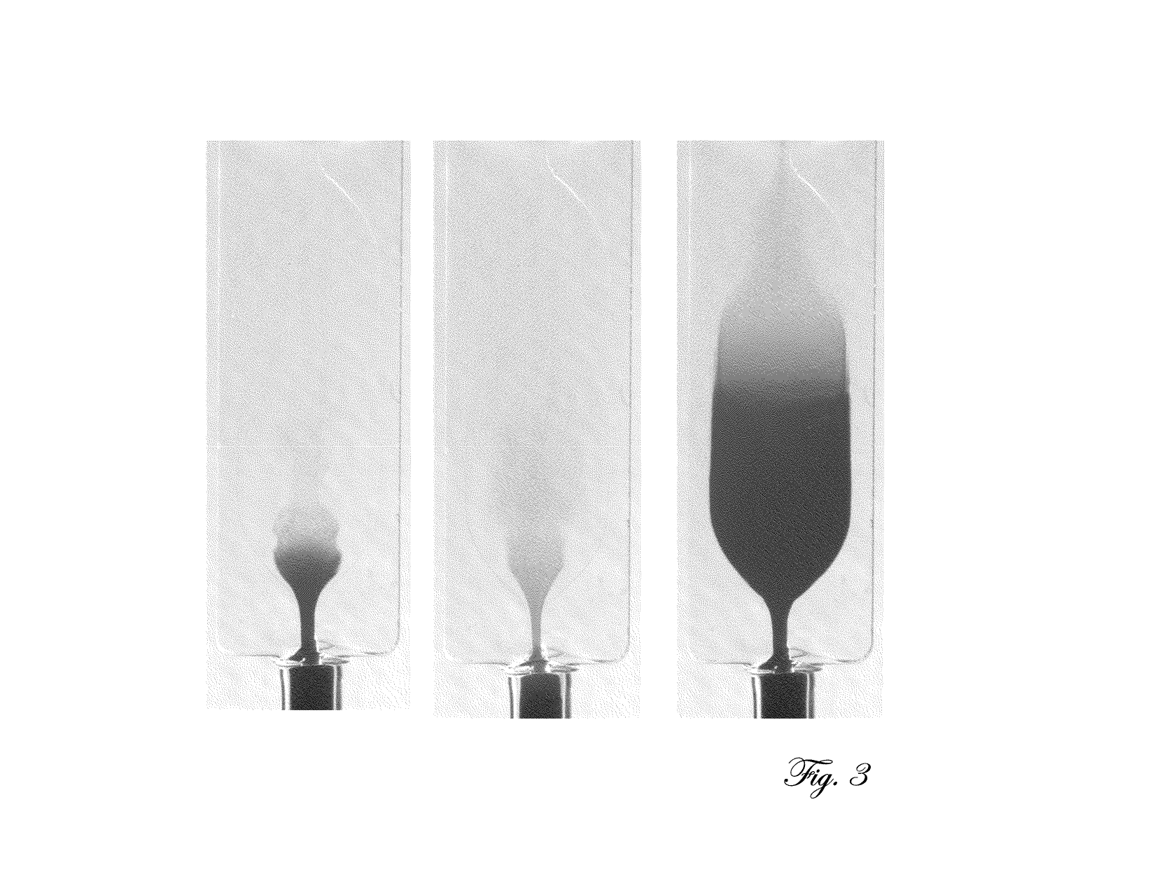

The difficulties in measuring the typical blood-saline interface are illustrated by FIG. 3 showing the various stages of blood entering a flow cell with an interactive flow field of blood in contact with saline.

Optical termination can be used to observe the sample composition during the blood draw stage; e.g., to decide when to start the measurement stage. Blood can be optically distinguished from saline because it has a distinct absorption in the visible through infrared spectral regions. Blood contains the distinguishable component absorbers of water (with a concentration less than pure saline), proteins (plasma and red blood cells biological matrix), and glucose. Blood also scatters light due to the 5-10 micron diameter red blood cells, which have a higher refractive index than the clear to yellow serum containing the cells. A robust real-time optical termination algorithm can compensate for blood composition changes across patients and treatments (e.g. transfusions), and blood optical changes with flow rate and temperature.

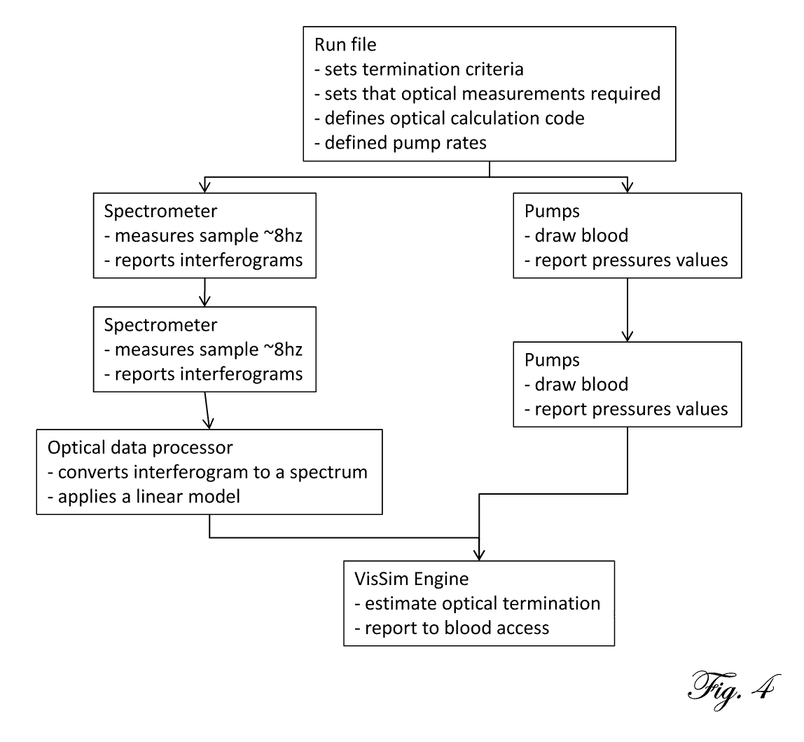

A demonstration of this concept is provided in the flow diagram in FIG. 4. This flow diagram shows a process by which the stages of blood sampling, optical measurement system (spectrometer), and blood draw system (pumps), interact to draw and measure the portion of blood in any flowing mixture of saline and blood.

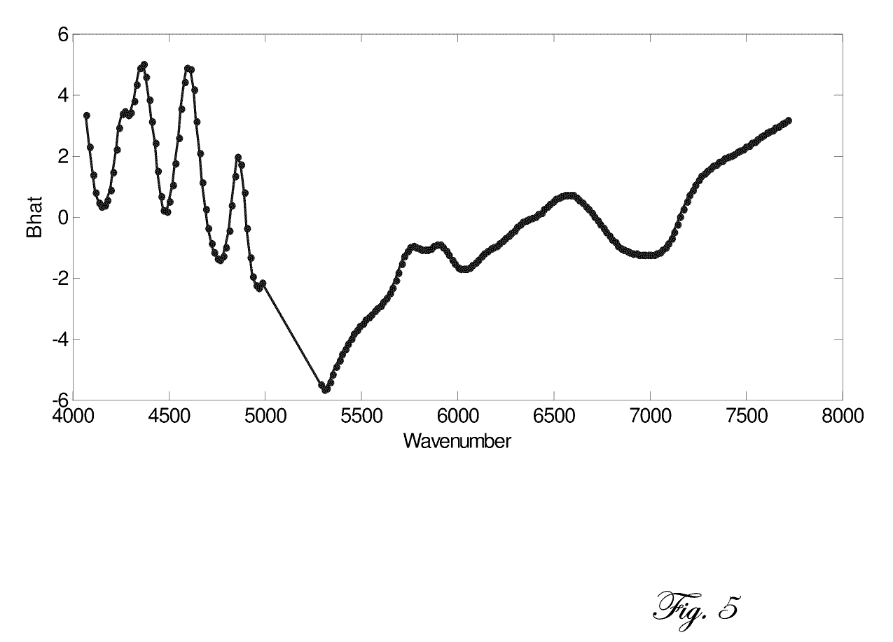

Optical termination of the blood draw has two linked algorithmic steps: (1) estimate the sample composition during each scan of the blood draw stage, and (2) Decide when to stop the rapid blood draw and start the measurement stage. This approach has been developed and tested on the example measuring system. The spectral model is designed to estimate ‘blood protein’ concentration, which is predominantly hemoglobin and plasma proteins. This predictor is designed to be insensitive to spectral features of the saline background including flow cell temperature and spectrometer changes (background slope and offset). The linear predictor (Bhat) was calculated with classic least squares and is illustrated in FIG. 5. This linear predictor is applied to the sample spectrum (dot product) to estimate the protein concentration. This algorithm emphasizes protein features in the 4000-5000 cm-1 spectral region.

The mathematical details associated with Bhat are presented in the software (Matlab) script for optical termination Bhat algorithm listed below.

% Predict blood concentration for optical termination
%
clear
%===================================================
% DEFINITIONS
%===================================================
% *** Model Building ***
pureCompFile = ‘BloodComponents.mat’;
CompInd = [6,1,2,7,8];
wRng = [4080,7700; 4975,5300];
% *** Model Testing ***
testFile = ‘Y:\Hyperion\CCP Studies\Prove-
in\DilutionMap\D001\formrgmt\dilutionmap+d001+cp1+smp+4.sgb’;
% *** Test Interferogram files ***
testIfg = ‘Y:\Hyperion\CCP Studies\Prove-
in\DilutionMap\D001\raw\dilutionmap+d001+cp1+smp+4.ifg’;
%===================================================
% CALCULATIONS
%===================================================
% *** Load spectral data ***
clc
disp([‘Loading pure components from ’, pureCompFile])
load(pureCompFile)
% *** Calculate predictors ***
disp(‘==================’)
disp(‘ Components used')
disp(‘==================’)
disp(Nblood(CompInd,:))
wi = SetIndex(wRng, wblood);
BhatAll = pinv(Ablood(CompInd,wi));
BhatBlood = BhatAll(:,1);
% *** Load spectra test Data ***
disp(‘ ’).
disp([‘Test data: ’, testFile])
[SgbTest, wTest] = readrgmt(testFile,‘off’);
wiTest = SetIndex(wRng, wTest);
Atest = −log10(SgbTest(:,wiTest));
Vtest = (1:size(Atest,1)) * 2/(8*60); %(2mL/min) /(8 scans/s * 60 s/min)
% *** Predict Blood Concentration ***
CbloodRaw = Atest * BhatBlood;
CbloodBC = CbloodRaw - mean(CbloodRaw(1:40,:));
% *** Load interferograms to test full spectra range predicitons ***
Ifg = readrgmt(testIfg);
[SgbFull, wfull] = ifg2sgb(Ifg, [0, 15791]);
Afull = −log10(SgbFull);
% *** Zero fill the predictor model for unused wavelengths***
wiFull = SetIndex(wRng, wfull);
BhatBloodFull = zeros(size(wfull,2),1);
BhatBloodFull(wiFull) = BhatBlood;
% *** Predict full spectrum ***
CbloodRawFull = Afull * BhatBloodFull;
CbloodBCFull = CbloodRawFull - mean(CbloodRawFull(1:40,:));

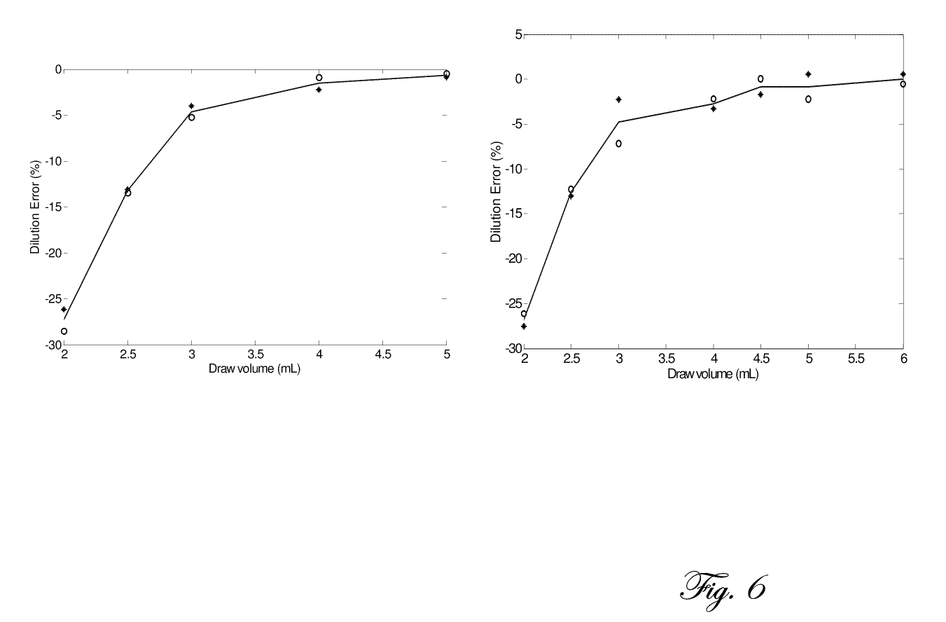

To test the method, an experimental protocol was developed including: (1) the drawing of human blood from a stirred beaker at 2 ml/min constant flow rate, (2) a stop pump function to operate at a set volume, (3) blood sample collection from the flow cell, (4) collection of a control sample from the beaker, (5) a measurement of the glucose values in duplicate on a reference glucose analyzer (i.e., YSI), and (6) repetition of the procedure at increased test draw volumes. FIG. 6 demonstrates the accuracy of the optical termination algorithm versus YSI glucose measurements on a saline-blood mixture. These results were generated with blood samples taken from the flow cell and measured on the YSI glucose analyzer. The grid lines are added at a 4 mL draw volume for reference and demonstrate acceptable accuracy for the algorithm termination versus the peak glucose concentration of the sample determined via the YSI reference method. The saline solution used does not contain any glucose.

For the actual termination of the draw sequence and the start of the measurement cycle a rate of change associated with the “blood protein” concentration is used. Since the amount of blood protein can change between people, the rate of change measurements have proven to be more robust measurement. As can be seen from FIG. 6, the blood concentration changes with increasing draw volume mirror an increasing exponential curve. Thus, a variety of metrics exist for determining then the rate of change has decreased to a reasonable level.

### Monitoring Sample Parameters During Sample Procurement or Measurement

Monitoring of temporal variability in a sample as it is either procured for measurement or as the analyte determination is made can provide an indication of sample stability. A system that incorporates a sensor capable of monitoring a sample parameter indicative of sample stability or quality during acquisition can provide an indication of the quality of the sample during procurement and/or analyte determination. The sensor can comprise the system used to measure the analyte or can comprise a separate sensor. FIG. 7 is a schematic illustration of an example system that incorporates a parameter sensor to evaluate sample quality during acquisition or measurement of a biological sample. Measurement of instability in sample properties or analyte concentration during sample acquisition prior to measurement or instability during the measurement can be used to automatically identify suspicious samples at the time of acquisition or measurement. With this information, the instrument can algorithmically evaluate the type and magnitude of variability, make a determination of sample suitability, and, if appropriate, inform the operator that the sample is suspicious or invalid, depending on predetermined thresholds and rules.

A variety of sample characteristics can be monitored to evaluate sample stability and quality during acquisition and measurement. Specific examples include:

Direct or indirect measurement of the concentration of the analyte of interest (e.g., glucose). Examples of methods to measure the analyte of interest include spectroscopic methods (transmission spectroscopy, diffuse reflectance spectroscopy, or transflectance spectroscopy in the ultra-violet, visible, near-infrared or mid-infrared regions; fluorescence spectroscopy using an analyte-sensitive fluorophore).
Direct or indirect measurement of another analyte in blood other than the analyte of interest that should yield consistent values in a stable sample (e.g., blood chemistry analytes such as sodium ion (Na+), chloride ion (Cl−).
Direct or indirect measurement of hematocrit.
Direct or indirect measurement of hemoglobin.
Direct or indirect measurement of plasma protein.
Direct or indirect measurement of sample temperature.
Direct or indirect measurement of sample optical density.
Direct or indirect measurement of sample optical scattering characteristics.

In addition, system characteristics can be monitored during sample procurement or measurement to provide an indicator of sample quality. For example, it can be desirable to maintain a controlled flow rate during the measurement of the sample analyte or parameter. Sensors that are used to monitor the flow rate of the sample can provide an indication of how well the system is controlling the sample draw characteristics during sample acquisition and measurement. Sensor output signals can be monitored to detect deviations from desired or targeted flow profiles, enabling the system to identify suspicious or problematic sample procurement and alert the user that a suspicious or faulty sample has been collected. Depending on the specific characteristics of the sample acquisition, the system can provide an alert to the user that a suspicious sample has been drawn and then, subject to pre-determined rules, the system can take appropriate action (for example, the system can automatically re-initiate the sample or can transition to a safe state and then cease further operation until the system is checked by the user). System parameters that can be monitored to provide useful information about system control of sample acquisition include, but are not limited to, the following examples:

System flow rate (direct monitoring of the sample flow rate in the tubing through which the sample is flowing during blood sample acquisition, for example). A variety of methods can be used to monitor the flow rate, including ultrasonic methods.
System pressures in the blood withdrawal line; such changes can indicate a change in line status such as a partial or total occlusion.
System pressures at two points within the blood withdrawal line, which can be used to evaluate changes in flow rate assuming that the viscosity of the sample is not changing during the sample phase of the withdrawal.
Air bubble detectors in the tubing through which the sample is being drawn.
Determination of Sample Quality based on Parameter Variation Profile

Sensors used to monitor a sample parameter in a continuous or near-continuous manner during procurement or measurement will generally have a signal response that is well-behaved provided the sensor is functioning properly and that a stable sample is being procured. The signal will generally have a mean value and will have some variance in the measured parameter that can be dependent on a variety of factors (including, as examples, sensor noise response characteristics, sensor sensitivity to the measured parameter, environmental conditions, etc.). Importantly, the sensor signal response can also depend on time-varying changes in the sample characteristics with regard to the measured parameter.

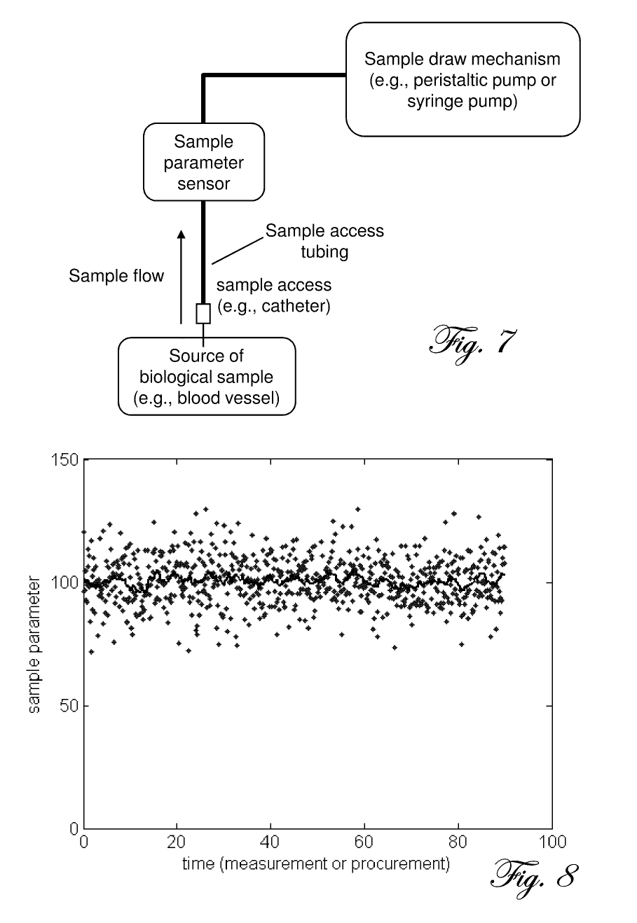

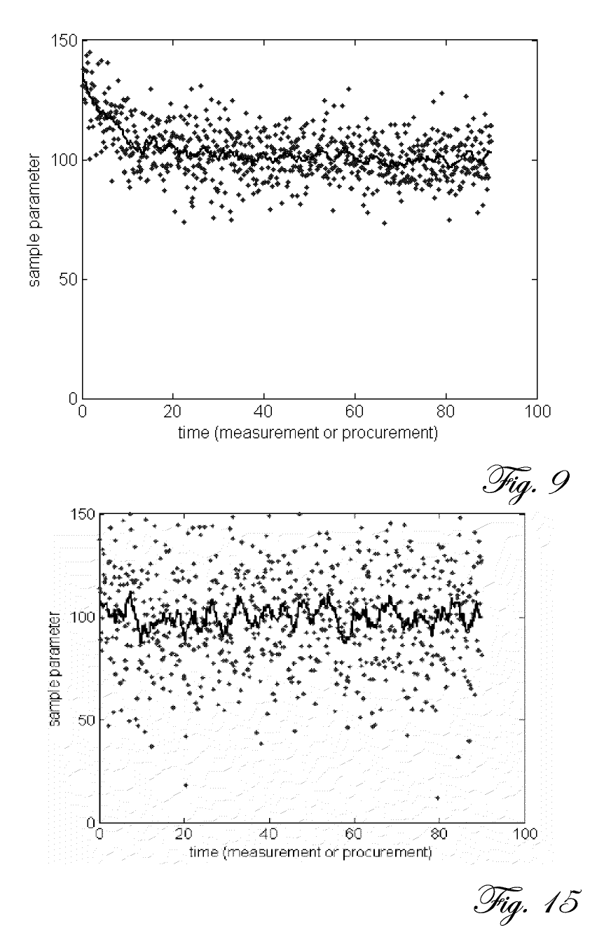

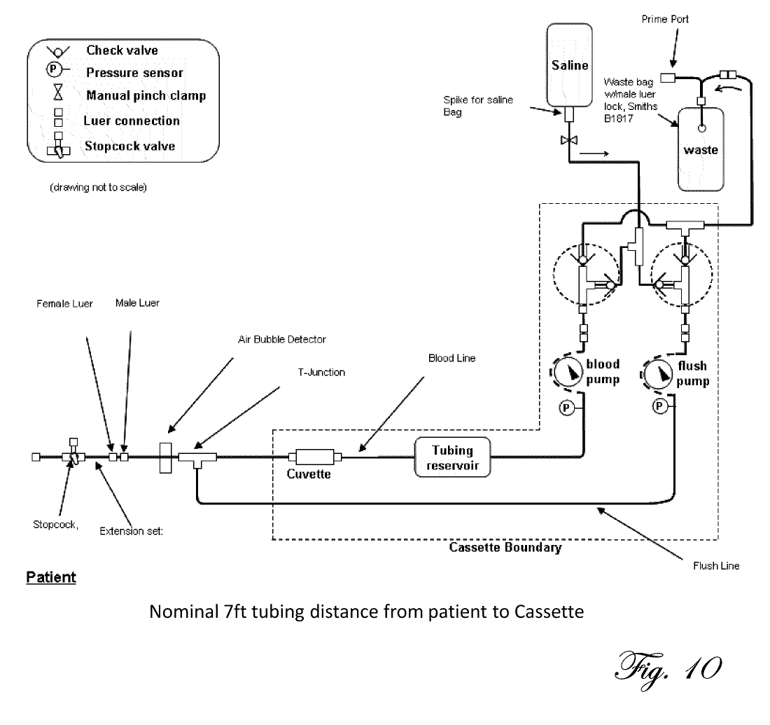

For example, a sensor that is sensitive to analyte concentration will respond to changes in the analyte concentration in the sample as the sensor is exposed to the time-varying sample. FIG. 8 is a plot of an example of a stable sample, in which the sensor response has a mean value with a characteristic noise profile in the discrete measurements of the sample parameter over the time course of the sample procurement. A low-pass filter can be designed and applied to the signal to filter out the high frequency stochastic noise associated with the sensor output variation while retaining information about the time-varying characteristics of the signal. FIG. 9 is a plot of an example of an unstable sample in which the measured sample parameter is varying in a trending fashion in the early phase of sample procurement. The low-pass filter response plotted on the figure emphasizes the time trend present in the measured parameter.

One approach to identifying samples with poor quality is to compare the overall variance of the sample to ensure that it is within a threshold that represents normal variation. This method will readily detect samples with high variance introduced by a significant change in the sample parameter sensor response, indicating a suspicious or faulty sample.

Another approach to identifying samples with unusual temporal variance profile entails applying a low-pass filter or moving average filter to the measured data (y) to obtain a reduced-noise representation of the temporal profile of the monitored parameter (y_{fit}). This temporal profile can then be subtracted from the original measurements to obtain a residual signal (y_{residual}). The variance of the residual signal can then be calculated and compared with the variance of the original data. A significant reduction in the variance of the residual data compared with the original data can be indicative of an unstable sample with significant temporal variation that is inflating overall signal variance.

### Detailed Description of Blood Access Operation in an Example Embodiment

The Blood Access System operation comprises 6 primary stages:

Draw initialization and clearing the catheter access;

Blood withdrawal;

Optical measurement;

Infusion;

Cleaning (incorporating Scrub, Recirculation, and Catheter Flush sub-stages);

KVO (“keep vein open”).

Draw Initialization Stage; Clearing Catheter Access. Before the blood draw is started, both the blood and flush pumps are controlled to issue a pulse of saline to clean away any residual blood in the catheter tip. This prepares the catheter for the subsequent withdrawal of blood.

Blood Withdrawal Stage. The blood pump is used to withdraw the blood sample and position non-diluted blood in the cuvette. To minimize the total draw time, about 80% of the total required blood volume is first drawn at a rapid flow rate. A constant-pressure-based draw method is used to compensate for the varying mix of saline and blood, and to achieve maximum flow rate constrained by the constant upstream negative pressure that keeps fluid degassing minimized. As blood replaces saline in the blood line, viscosity and resistance to flow increase so that for a constant upstream pressure, flow rate decreases over time. The termination of this stage of the draw is determined by what is referred to as optical termination. Optical termination is the optical detection of when a sample appropriate for measurement has filled the cuvette. After the optical termination of the withdrawal stage, the measurement of the sample can be initiated. An example of a specific optical termination method will be disclosed in detail below. Non-optical methods of detecting the arrival of an undiluted blood sample, such as those described elsewhere herein, can also be used.

Optical Measurement Stage. Following the rapid draw, the pump flow rate is slowed to a constant flow rate of 0.5 mL/min to maintain suspension of the red blood cells in plasma during optical measurement. During the 60 second measurement period an additional 500 μl of blood is withdrawn.

Infusion Stage. After the measurement is completed, reinfusion immediately begins as a progression of stages that are designed to return the blood quickly to the patient and clean the tubing and optical cuvette. The initial stage of infusion uses a constant pressure-based control which results in a variable flow rate that minimizes the time to reinfuse the blood to the patient. This stage reinfuses nearly all of the blood that was withdrawn, leaving a remaining saline-blood mixture at the end of the blood line. The first stage of the reinfusion can be completed within three minutes of the initiation of blood withdrawal.

The 2nd stage of infusion involves a repetitive back and forth motion of the blood pump such that during half of one cycle the pump pushes blood forward at a constant flow rate, and during the second half of the cycle blood is pulled back at about half the rate. The asymmetric cycle helps wash away any cells or other blood products that could potentially adhere to the tubing walls. During this stage of infusion, flow is controlled to limit the pressure.

The 3rd stage of infusion begins with the blood pump executing a repetitive alternating forward-pause motion that provides pulsatile acceleration and washing of blood products from the tubing walls. The flow in this stage is also pressure controlled.

It is possible to use another optical termination type measurement to determine when the majority of blood has been re-infused back into the patient and exited the optical cell. The basic principles are the same but in this application the termination measurement is looking for stability in the saline sample instead of stability in the blood sample. The method can be used to make sure there is no residual blood in the cell.

Cleaning Stages. At this point in the cycle more than 97% of the blood has been returned to the patient; the next stages focus on a more thorough cleaning of the cuvette, tubing and catheter.

Scrub Stage. The first stage of cleaning is known as ‘scrub’. The scrub stage involves rapid, reverse-synchronized back and forth motion of the blood and flush pumps so that fluid movement occurs only within the blood and flush lines with minimum net fluid flow to or from the patient. The flow is not turbulent, but the rapid oscillations create accelerations that help to wash any small amount of residual blood that can collect on the walls of the tubing and cuvette.

Recirculation Stage. Once the remaining blood products have been lifted off the tubing and cuvette walls into the mainstream by the scrub stage, the blood and flush pumps are operated at a constant, nearly synchronized rate, flushing the lines into the waste bag while flushing a small amount of saline to the patient to keep blood from migrating back into the catheter. It is also possible to use an optical termination type measurement to access when the cell has been adequately cleaned. Even small amounts of protein can be assessed optically. Thus, the optical measurement method can be used to determine when adequate cleaning of the cell has occurred. In use the method can compare the spectral response from a prior measurement to the current measurement. If there is optical evidence of additional protein in the cell then additional cleaning might be indicated.

Catheter Flush Stage. In the final cleaning stage high flow rate controlled volume pulses completely clear the catheter extension line, tubing connectors and the catheter itself.

KVO Stage. The period between measurement cycles is KVO (Keep Vein Open). KVO provides a low, constant flow rate into the patient to prevent blood from migrating into the catheter thus maintaining an open blood access connection between draws.

Example Measurement Sequence with Measurement Quality Determination

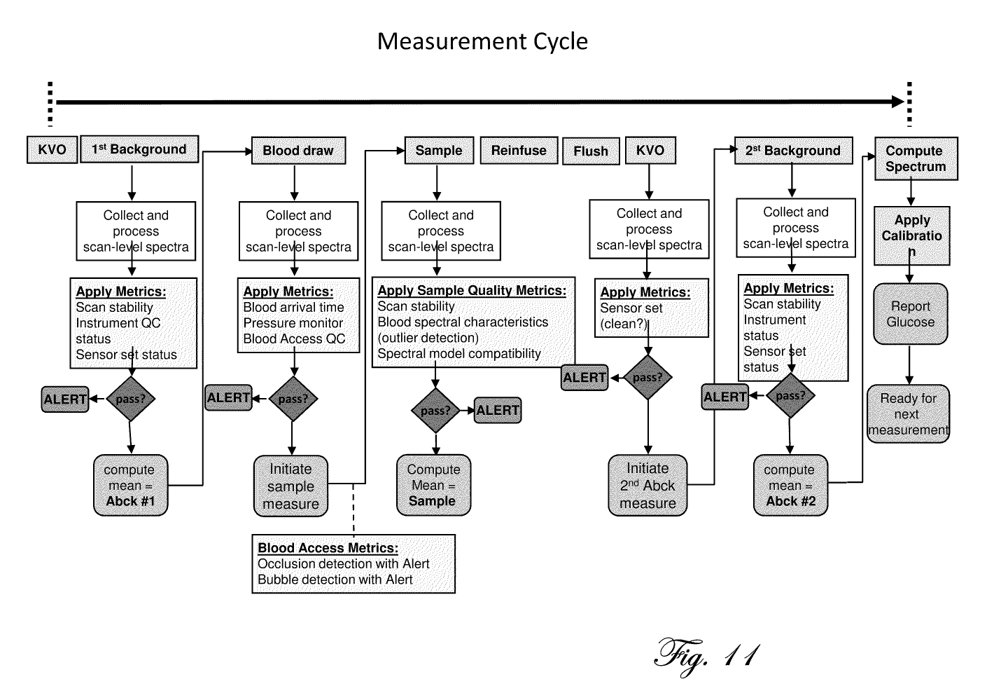

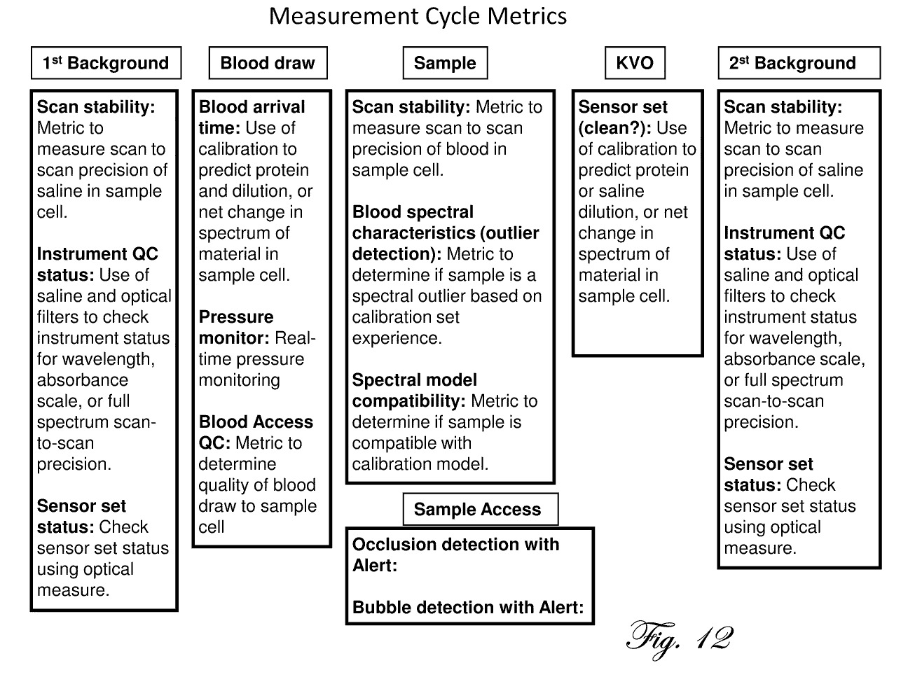

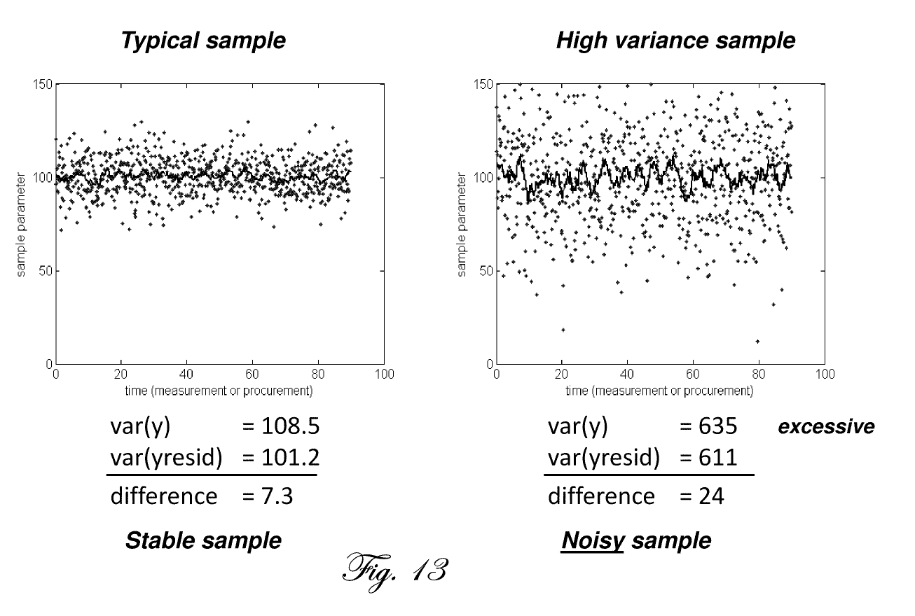

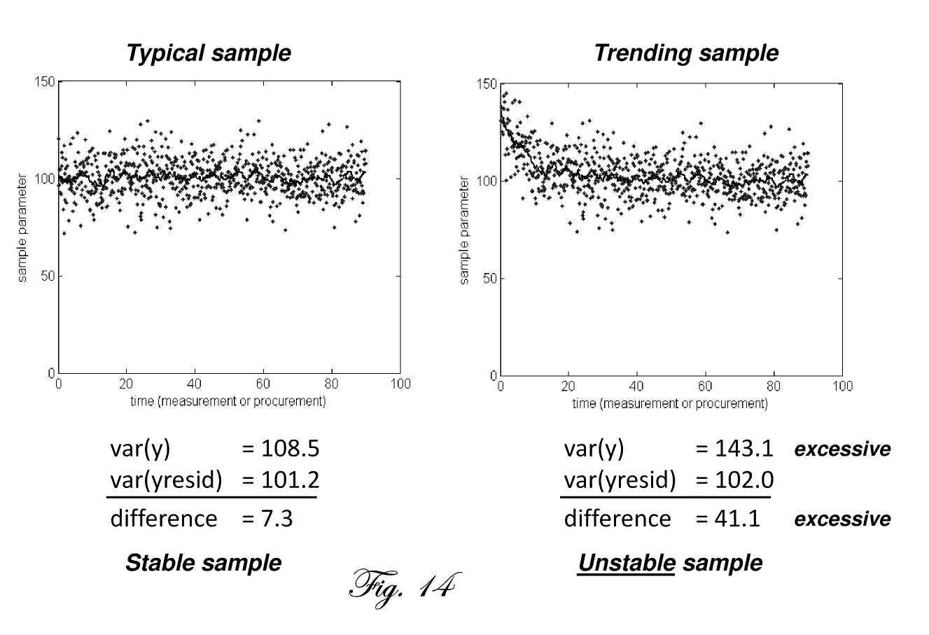

FIG. 11 provides a block diagram of the measurement sequence for an automated blood glucose monitor as described in the preceding section. During each phase of the measurement cycle, various parameters are monitored to determine proper operation and functionality of the system. An overview of the parameters used to monitor the system and the sample are indicated in FIG. 12.

In the “1st Background” phase, measurements can be taken of the fluid present at the measurement site, which fluid should be primarily saline (or other system fluid, and not blood). The measurements can be analyzed for variance and trends as described elsewhere herein. If the variance and trends do not match those expected for this phase of operation, then an error can be indicated.

In the “Blood draw” phase, measurements can be taken of the fluid that is present at the measurement site, which fluid should be transitioning from primarily saline (or other system fluid) to a mix of saline and blood to blood with minimal saline. The measurements can be analyzed for variance and trends as described elsewhere herein. As examples, any parameters that are present differently in blood than in saline (e.g., optical scatter, or some analyte concentrations) should show a time trend from the saline value to the blood value, then become stable after the measurement site is largely filled with blood. If the measurements do not indicate that the fluid is transitioning to substantially pure blood, then an error can be indicated.

In the “Sample” phase, the measurement site should be exposed to substantially pure blood sample. Measurements taken should show variance and stability consistent with such a sample, e.g., generally little or no trends, and variability within the range established by the measurement system itself. If the measurements are not consistent with a substantially pure blood sample, then an error can be indicated.

In the “Reinfuse”, “Flush”, and “KVO” phases, the measurement site should be exposed to varying combinations of blood and saline, ending with substantially pure saline by the KVO phase. Measurements taken during these phases should have trends and variability consistent with a declining proportion of blood present at the measurement site. If they do not, then an error can be indicated.

In the “2^{nd}

### Example Embodiments

FIGS. 8, 9, and 15 comprise plots of a sample parameter exhibiting three different overall characteristics. The parameter can be determined in various ways, for example using an optical measurement system, or using an electrochemical measurement sensor, or using an ultrasound sensor. The parameter can comprise a single property of the sample, or a combination of properties. The parameter used for quality assessment can be the same parameter as that desired to be measured, or can be a different parameter that can serve as an indicator of the quality of the desired parameter measurement. The parameter used for quality assessment can be measured using the same sensor as used for the parameter desired to be measured, or can be measured using a different sensor system.

FIG. 8 is a plot of a parameter used to assess quality, where the parameter does not exhibit significant time trends or variability greater than that expected for the parameter and sensor used. For example, the parameter can comprise concentration of an analyte, in which case the plot indicates that the analyte concentration is stable over time and has a value near 100. As another example, the parameter can comprise a measurement of sample temperature or optical scattering, while the parameter of interest is concentration of an analyte. In this case, the plot indicates that the temperature or optical scattering measure is stable over time, indicating that the sample present for analyte concentration measurement is stable and the corresponding analyte measurement is likely to be accurate.

FIG. 9 is a plot of a parameter used to assess quality, where the parameter shows a decreasing value over time (also referred to as a “trend” or “time trend”). For example, the parameter can comprise concentration of an analyte, in which case the plot indicates that the analyte concentration is decreasing over time and approaching a stable value of about 100. This analysis can be used to indicate when an acceptable sample measurement has been made, i.e., when the time trend decreases and leaves a stable value. As another example, the parameter can be a measurement of sample temperature or optical scattering, in which case the plot indicates that the sample is changing over time, for example as the sample presented to the measurement system changes from saline to blood/saline mix to blood. Measurements of the desired blood property can be determined to be inaccurate while the dilution is changing, as indicated by the time trend of the sample quality parameter.

FIG. 15 is a plot of a parameter used to assess quality, where the parameter does not exhibit a significant time trend but does exhibit variability greater than the expected range for the parameter and sensor. As an example, the parameter can be concentration of an analyte in the sample, and the variability can indicate that the sensor system is not operating in acceptable performance limits. As another example, the parameter can be a measurement of sample temperature or optical scattering, in which case the excessive variability can indicate that the system has presented an unacceptable sample to the analyte measurement system, and the accuracy of the analyte measurement can be in question. This can be important if the nature of the excessive variability can lead to inaccurate but stable analyte measurement, so analysis of the analyte measurement itself might not reveal the error.

The particular sizes and equipment discussed herein are cited merely to illustrate particular embodiments of the invention. It is contemplated that the use of the invention can involve components having different sizes and characteristics. It is intended that the scope of the invention be defined by the claims appended hereto. The above description discloses several methods and materials of the present invention. This invention is susceptible to modifications in the methods and materials, as well as alterations in the fabrication methods and equipment. Such modifications will become apparent to those skilled in the art from a consideration of this disclosure or practice of the invention disclosed herein. Consequently, it is not intended that this invention be limited to the specific embodiments disclosed herein, but that it cover all modifications and alternatives coming within the true scope and spirit of the invention.

What is claimed is:

1) In a system comprising a blood access system that draws blood from a patient to a measurement site through a passageway that contains a fluid wherein the blood and fluid can combine, a method of determining when the measurement site is exposed to a combination of fluid and blood that is suitable for making a measurement, comprising determining a characteristic of the fluid/blood combination that is located a determined distance from the measurement site, and determining from that characteristic whether the fluid/blood combination at the measurement site is suitable for making a measurement.

2) A method as in claim 1, wherein determining a characteristic of the fluid/blood combination comprises determining at least one of optical density, optical scatter, analyte level, temperature, the absolute level of any of the preceding, the stability of any of the preceding, the rate of change of any of the preceding, or the value of any of the preceding relative to another determination.

3) A method as in claim 1, wherein the determined characteristic comprises at least one of: optical density, optical scatter, analyte level, temperature, the absolute level of any of the preceding, the stability of any of the preceding, the rate of change of any of the preceding, or the value of any of the preceding relative to another determination.

4) A method as in claim 1, wherein the characteristic is determined using at least one of: absorption spectroscopy, Raman spectroscopy, fluorescence spectroscopy, atomic absorption spectroscopy, attenuated total reflectance spectroscopy, electron paramagnetic spectroscopy, electron spectroscopy, gamma-ray spectroscopy, infrared spectroscopy, laser spectroscopy, mass spectrometry, and x-ray spectroscopy.

5) A method as in claim 1, wherein the characteristic is determined using at least one of: electrochemical sensor, ion specific electrode, capacitance measurement, impedance measurement, inductance measurement, conductivity measurement, optical measurement, and ultrasound measurement.

6) A method of determining a first characteristic of blood withdrawn from a patient, comprising withdrawing blood into a channel that contains a fluid by drawing the fluid away from the patient, measuring a second characteristic of the fluid/blood combination in the channel wherein the characteristic is related to the relative proportions of fluid and blood in the fluid/blood combination, determining when the second characteristic indicates that the proportion of blood in the fluid/blood combination is suitable for a determination of the first characteristic, and then determining the first characteristic.

7) A method as in claim 6, wherein the first characteristic is the concentration of glucose in the blood.

8) A method as in claim 6, wherein the second characteristic is at least one of: optical density, optical scatter, analyte level, temperature, the absolute level of any of the preceding, the stability of any of the preceding, the rate of change of any of the preceding, or the value of any of the preceding relative to another determination.

9) A method as in claim 7, wherein the first characteristic is determined from the response of the fluid/blood combination to optical energy.

10) A method as in claim 7, wherein the first characteristic is determined using an electrochemical sensor.

11) A method as in claim 6, wherein the second characteristic is determined using at least one of: electrochemical sensor, ion specific electrode, capacitance measurement, impedance measurement, inductance measurement, conductivity measurement, optical measurement, and ultrasound measurement.

12) A method as in claim 6, wherein determining the first characteristic comprises operating a sensor for a time determined based on the second characteristic.

13) An apparatus for the determination of a property of blood withdrawn from a patient, comprising:
a) A blood access system, comprising tubing adapted to transport blood from a patient and a pumping subsystem adapted to urge fluid in the tubing toward and away from the patient;
b) A measurement subsystem mounted with the blood access system and adapted to determine first and second properties of fluid in the blood access system;
c) An analysis system adapted to determine from the second property whether the fluid accessible to the measurement system is suitable for a determination of the first property.

14) An apparatus as in claim 13, further comprising a source of transport fluid in fluid communication with the blood access system, and wherein the second property is indicative of the mixing of blood with the transport fluid.

15) A method for determining the quality of a biological sample procured for ex vivo analysis, comprising:
a) measuring a parameter of the biological sample at two or more distinct times;
b) analyzing the measurements to determine a relationship between the two or more measurements;
c) determining whether the relationship within predetermined limits.

16) A method as in claim 15, wherein the relationship comprises a time-dependent profile.

17) A method as in claim 15, wherein the relationship comprises the variability of the two or more measurements.

18. A method as in claim 18 wherein the measured parameter is glucose concentration.

19. A method as in claim 15 wherein glucose is measured by near-infrared spectroscopy, near-infrared transmission spectroscopy, mid-infrared transmission spectroscopy, raman spectroscopy, fluorescence spectroscopy, absorbance spectroscopy, or a combination thereof.

20. A method as in claim 18 wherein glucose is measured by an electrochemical sensor or an enzymatic sensor.

21. A method as in claim 15 wherein the measured parameter is hematocrit, protein, water concentration, hemoglobin, fluid flow, sample temperature, or a combination thereof.
